(19) United States
(12) Patent Application Publication
(10) Pub. No.: US 2016/0376543 A1
(43) Pub. Date: Dec. 29, 2016

(54) METHOD OF CULTURING ALGAE

(71) Applicant: Phycotechnology Solutions, LLC, Houston, TX (US)

(72) Inventor: Timothy F. Hall, Houston, TX (US)

(21) Appl. No.: 14/752,549

(22) Filed: Jun. 26, 2015

(51) Int. Cl. C12N 1/12

(52) U.S. Cl. CPC C12N 1/12

(57) ABSTRACT

Described are culture media for growing algae. Also described are methods for preparing an algal culture media and methods for culturing algae.

### FIELD OF THE INVENTION

The disclosure relates to aquaculture and methods for culturing algae, including microalgae, and methods of increasing yield of algae product. Also disclosed is a culture medium for growing algae in varying environmental conditions.

### BACKGROUND OF THE INVENTION

Algae are a diverse group of photosynthetic organisms that typically live in aquatic environments. They range in size from microalgae, which can be only microns in size, to macroalgae—such as giant kelps—that can be up to 60 meters long. Microalgae, or “microphytes,” are commonly found in freshwater and marine systems. In the oceans, they are primary producers for converting water and carbon dioxide to biomass and oxygen in the presence of sunlight. Microalgae have a long history for use as a renewable feedstock and have received significant recent attention as a potential source of renewable energy. Thousands of years ago, the Chinese used microalgae as a food source to avoid starvation. However, it was not until later that scientists first began serious consideration of large-scale cultivation of microalgae to derive other useful products (Milledge, 2010).

In recent years, microalgae has generated significant attention because of its increased productivity (e.g., reproductivity rates, renewability rates) relative to other biomass feedstock. Unlike plants, nearly 100% of the biomass of microalgae can be used in certain applications. Algae biomass can be directly used in agricultural and bioremediation applications. In addition, algae derivatives hold significant promise in the nutraceutical, pharmaceutical and bioenergy industries. Interest in third-generation biofuels has increased for mass culture of algae for the creation of biomass for oil and lipid extraction (Oswald & Goluke, 1960).

Algae can be grown using water resources that are otherwise unsuitable for cultivating agricultural crops, including brackish-, sea-, and wastewater. Wastewater includes municipal, animal and industrial runoff. In such conditions, algae can be useful in treating and purifying the wastewater, while benefiting from the nutrients present therein.

Industrial algae production commonly involves the use of commercial-scale open ponds and circular open-air water treatment plants. These open ponds are inexpensive, relatively easy to maintain, and allow production of significant quantities of algal biomass. However, this method is limited because of evaporation, susceptibility to contamination, and other hostile factors. These systems are also limited by problems with mixing and inconsistent gas (e.g., oxygen and carbon dioxide) exchange. Many operators now use oblong raceway ponds with paddlewheels to circulate the water and growing algae. Though raceway ponds have enabled researchers to generate significant quantities of microalgal biomass, this system is still limited because of the microalgae's sensitivity to contamination, evaporation, micronutrient, gas and pH variability. Algae grown in open ponds are also susceptible to predation by organisms/microorganisms, microbial competition, and bacteria/virus infections. These and other factors contribute to the high frequency of lost culture viability (i.e., culture crash) in both large and small-scale algae culture. To date, no cost-effective alternative technology has been developed that overcomes these problems while maintaining the level of productivity seen in open systems.

### SUMMARY OF THE INVENTION

In one aspect, a conditioned culture medium for growing algae is disclosed. In some embodiments, the conditioned culture media comprises water obtained from a stock pond, wherein the water has been treated to release biotic and abiotic factors from the natural flora of the stock pond. In embodiments, phosphates and nitrates are added to the conditioned culture media. In certain embodiments, a dry conditioned culture medium is disclosed. Such a dry medium may be in powder or pill form, for easy addition to a basic culture medium.

In one aspect, methods for making an algal culture medium are disclosed. In some embodiments, the method comprises treating water obtained from a stock pond to release biotic and abiotic factors from the natural flora of the stock pond. In certain embodiments, the flora are lysed to release these factors.

In one aspect, methods for growing algae are disclosed. In some embodiments, provided are methods for culturing microalgae. In certain embodiments, provided herein are methods for growing microalgae in high yield, wherein the microalgae have improved resistance to culture crash and reduced viability. In certain embodiments, methods for growing algae comprise treating water obtained from a stock pond to release biotic and abiotic factors from the natural flora of the stock pond. In certain embodiments, the flora are lysed to release these factors.

### BRIEF DESCRIPTION OF THE DRAWINGS

FIG. 1 is a process diagram showing one embodiment of a method for culturing algae, as disclosed herein.

FIG. 2 is a graph illustrating algal cell growth (cells per mL) using conventional F2 culture media (AF-F2) and conditioned culture media (TH-F2).

FIG. 3 is a graph illustrating algal cell growth (cells per mL) using conventional F2 culture media (AF-F2) and conditioned culture media (TH-F2) over four days.

FIG. 4 is a graph illustrating algal cell growth in terms of optical density (OD) in relation to culture pH over time, using conventional F2 culture media (AF-2) and conditioned culture media (TH-F2) over several days.

### DETAILED DESCRIPTION OF THE INVENTION

Existing methods for culturing algae rely on the assumption that the algal organism can be removed from its natural environment, isolated in a laboratory or culture pond, and raised on an engineered food source, without a significant effect from the loss of the dynamic biochemical factors and symbiotic relationships that promote growth in the wild.

Thus, significant research is directed at formulating “complete” algal culture media that can sustain an algal culture. These culture media begin with homogeneous aseptic ingredients that are mixed with water. The aseptic technique provides (1) a very specific type of medium carefully designed to promote cellular viability, and (2) a medium devoid of contaminants, especially microbial organisms such as bacteria, viruses, and spores, that could cause the culture to lose viability. However, no matter how carefully prepared the medium is, it cannot duplicate adequately the in situ growth characteristics provided by the biochemical interactions in the natural environment.

The present applicant has found that removal of the algal organism from its natural environment alters its growth characteristics, despite careful attempts to culture it ex-situ. Natural symbiotic relations with other flora and biochemical interactions present in the natural setting become disrupted. More energy is thus required to support the organism in a viable state than is practically captured for useful biomass production; this contributes to an increase in mutations and/or a loss of viability of the organism in the culturing process. These problems are not addressed effectively by supplementation with traditional culture media.

Methods for growing algae, as disclosed herein, are associated with creating a controlled growing environment for the algae that more closely mimics the organism's natural environment. The dislosure includes methods for preparing an algal culture medium that retains many of these endogenous (i.e., in situ) factors and interactions. The media is designed to minimize the energy needed by the cell for survival/protection and to provide food sources that are readily available for cellular uptake and metabolic processes.

Also disclosed are methods for growing algae, including methods that use the aforementioned algal culture media. Methods for growing algae disclosed herein are associated with improved resistance of the microalgae culture to a loss of viability, generally referred to as “culture crash.” These methods and the culture media disclosed herein provide other advantages over traditional algal culture methods, as discussed further herein.

Abiotic factors, as used herein, refers to and includes various non-biologically derived factors that are generally necessary for or supportive of algal growth. These factors include, but are not limited to, minerals (e.g., iron, sulfur, phosphorous), including minerals bound in composition and ionic form (e.g., nitrates, sulfates, phosphates, etc.); light; temperature; salinity; pH; and water concentrations.

Aeration. Algae require a consistent source of carbon dioxide (CO₂) and they produce oxygen (O2). It has surpringly been found that culture of algae according to methods disclosed herein and/or using the conditioned culture media disclosed herein allows the user to culture algae without the need for continous bubbling of gases in a culture tank.

Algae, as used herein, refers to and includes any of a wide variety of simple plant-like eukaryotic organisms. The methods and culture media for growing algae, as disclosed herein, are suitable for any type of freshwater or marine algae. The term “algae” has no formal taxonomic status. Algae are very diverse and are found almost everywhere on the planet (Barsanti, 2006). Most algae grow through photosynthesis—by converting sunlight, CO₂ and nutrients—including nitrogen and phosphorous—into biomass (“photo-autotrophic” growth). Other algae can grow in the dark using sugar or starch (“heterotrophic” growth), or even combine both growth modes (“mixotrophic” growth). Algae play an important role in many ecosystems, providing the foundation for the aquatic food chains supporting the fisheries in the oceans and inland, and producing over 60 percent of the air humans breathe. Algae includes both microalgae, which can be as small as 0.2-2.0 μm, and macroalgae, which can be several meters long.

In certain preferred embodiments, the culture media and methods disclosed herein are used in the culture of microalgae. Suitable microalgae include Botryococcus, Chorella sp., Dunaliella sp. (e.g., Dunaliella salina), Emiliania huxleyi (EHUX), Haematococcus sp., Nannochloropsis, Scenedesmus sp., Spirogyra sp., Spirulina, diatoms including bloom forming diatoms (e.g., Pseudo-nitzschia sp.), dinoflagellates (e.g., Alexandrium sp., Ceratium sp.), raphidophytes (e.g., Heterosigma akashiwo) and cyanobacteria (formerly called “blue-green algae”) (a prokaryotic form). Scenedesmus dimorphus, as used in some embodiments herein, is a freshwater unicellular algae in the class Chlorophyceae, one of the classes of green algae.

Biotic factors, as used herein, refers to and includes various biologically derived factors that are generally necessary for or supportive of algal growth. These factors include, but are not limited to, growth factors, hormones, amino acids, and proteins found in and/or produced by living organisms.

Cell density. The cell density of algae in some commercial operations, including open raceway ponds, can reach several million cells/ml in order to maintain a high standing stock of algae to supply a continuous stream that can be harvested effectively. The density of algae cultivated in raceway ponds is often high enough that the algae respond in deleterious ways, including culture crash. Culture crashes commonly occur as declining nutrient levels and water quality cause a sharp decrease in algal cell density and eventually culture collapse. Cultivation of algae according to methods disclosed herein and/or using the conditioned culture medium disclosed herein is associated with improved algal growth and maintenance of very high density (“super-concentrated”) algal cultures for prolonged periods of time without a loss of algal viability.

Conditioned culture medium. In some embodiments, provided is a conditioned culture medium for growing microalgae. The conditioned culture medium, sometimes referred to herein as PTS, more closely mimics the aqueous environment experienced by algae in nature, as compared to traditional or standard culture media commonly used to grow algae commercially. Commercially-available culture media for growing algae and microalgae have significant limitations that reduce their effectiveness for long-term applications. These include a failure to regulate pH, predators feeding on the algae, and labor-intensive care in maintaining algae cultures. As a result, culture crashes are frequently seen when algal cultures are maintained for prolonged periods on traditional media.

In some embodiments, a conditioned culture medium using natural pond water as a source of a fluid component of the media is disclosed. Natural pond water for use in preparing an algae culture medium may comprise preexisting algal and other biotic growth, including, e.g., small plants and other photosynthetic organisms, and larger organisms native to the habitat. In addition, water in a healthy natural pond contains many of the dynamic factors in an aqueous form that contribute to normal algal growth, in situ. A method for preparing a conditioned culture medium may include treating pond water to release various factors from the native flora in the water. These factors may include, for example, biotic factors such as growth factors, hormones, amino acids, and proteins, and abiotic factors, such as organic and inorganic cofactors and minerals associated with the native medium. This treatment is expected to release both internal cellular contents and membrane or vesicle-bound cellular components.

In some embodiments, the native flora in the pond water is treated to lyse the microbial cells present in the water. Various methods are known for lysis of cells, including methods intended to lyse algal cells themselves to harvest a desired algal cell product. These lytic methods include heat treatment using, for example, an autoclave; sonication; and application of detergents or other chemicals to disrupt cell membranes (US20120021481, WO2014027871), the contents of which are incorporated herein by this reference. In a preferred embodiment, heat treatment is used to lyse the cells in the pond water before the water is used to prepare a conditioned culture medium. Lysis of cells to harvest internal cellular contents is known in the art. However, the use of lysed cells from natural pond water to prepare an algal conditioned culture medium was not known or suggested in the art.

Autoclaving accomplishes two main goals: (1) eliminating predators and sources of infection/contamination, and (2) providing a rich source of biochemicals that can be readily taken into cells, minimizing energy expenditure. Autoclaving uses sufficient moisturized heat and pressure to lyse the cells of all the microorganisms and spores in the medium. This in turn releases all the cellular content of the cells: amino acids, hormones, growth factors, enzymes, and cofactors etc. With the removal of predators and sources of infection/competition, the algal cells, in such a rich source of nutrients, are provided a means for rapid cellular growth and reproduction at lower cellular energy costs.

In some embodiments, the conditioned culture media is further enhanced by the addition of biochemical factors found in the natural environment. In some embodiments, predators and viruses that could harm the growing algae are removed from the media or destroyed by, for example, filtration, chemical, heat, pressure, or light (radiation) treatment. In some embodiments, treatment of the pond water by, for example, heat treatment, may serve both to lyse the cellular components of the pond water, as described hereinabove, and to kill or otherwise disinfect the water. In other embodiments, both a lytic step and a filtration step may promote more effective disinfection or purification of the pond water before use in algal culture.

In some embodiments, the conditioned culture medium is dried or dessicated after preparation to form, for example, a powder or pill. The dry conditioned culture medium can be stored in suitable conditions to preserve the endogenous biotic and abiotic factors present in the medium, then subsequently added to water or basic culture medium at a preferred time when needed. Drying of the conditioned culture medium may be accomplished by dessication, heating, vacuum dehydration, or any other suitable drying method.

In some embodiments, a conditioned culture medium is used to rescue late-growth stage cultures from an imminent or ongoing “culture crash.” In certain embodiments, rescue of late-growth stage cultures using the disclosed conditioned culture medium restores viability and positive growth characteristics of the algal culture.

Environmental conditions. Methods for culturing microalgae and the culture media disclosed herein are suitable for growing microalgae in a range of environmental conditions. The algal response to external stimuli—including its response to salinity, temperature, nutrient, light or population dynamics—changes its physiology and behaviour. These responses depend on the intensity, frequency, and combination of these environmental factors. Optimal lighting may be provided by the use of heat lamps or other suitable light source, as known by those of skill in the art. As algal cell density increases, additional lamps are required to promote continued growth of the culture.

The optimal reported pH range for microalgae culture is approximately pH 8.2-8.7. An optimal pH may be determined by observing growth rates, sustainability, and reproductive capacity of the algae culture under varying acidity conditions. Culture pH may be adjusted as needed by addition of suitable acidic or alkaline agents (e.g., sulfuric acid). Most reports indicate that the pH of the culture should not be allowed to exceed pH 9.0. It has been discovered, however, that culture of algae according to methods disclosed herein and/or using the conditioned culture media disclosed herein allows algae to thrive at a wider range of pH values than seen with standard algae culture media alone, with little or no need for regular user intervention to maintain pH. It has surprisingly been found that—using the culture media and methods disclosed herein—microalgae can be successfully cultured and maintained at pH ranges of about between 9.0 and 10.0 for several months. In certain embodiments, the algal culture is maintained at about 10.0 for extended periods, without a noticeable loss of algal cell viability.

Microalgae products. Microalgae cultured according to methods disclosed herein may be used to produce a variety of useful byproducts, including biofuels (e.g., biodiesel) and algal-derived oils. Algae biofuel is an alternative to fossil fuel that uses algae as its source of natural deposits. Like fossil fuel, algae fuel releases CO2 when burnt, but unlike fossil fuel, algae fuel and other biofuels only release CO2 recently removed from the atmosphere via photosynthesis as the algae or plant grew. Unlike fossil fuels, algae are also a renewable resource without many of the environmental concerns associated with fossil fuel production

Recycling. In some embodiments, the method for culturing algae includes a recycling step. As algal cultures begin to increase in cell density, a semi-continuous, multi-step harvest process may be used to allow recovery of algae biomass. In certain embodiments, this recovery process may comprise sedimentation of the algae to form a solid or semisolid mass or aggregate, with recovery of the conditioned culture medium (PTS medium) from the algal mass, to allow continued use of the valuable medium

Water Source. In some embodiments, water from a stock pond is used as the source of the aqueous component of a culture medium disclosed herein. The stock pond may be either natural or man-made. Natural pond water is expected to contain a number or biotic and abiotic factors that contribute to successful algal growth in situ. Treatment of this natural pond water source according to methods disclosed herein can further release growth-enhancing substances that allow algae to thrive.

A mature stock pond has a well-established flora of microorganisms, and the water itself has absorbed and balanced many of the organic and inorganic chemicals of its environment. The use of natural water for making freshwater media is uncommon. Typically, the person of skill in the art uses artificial media with distilled water to culture algae.

The soil water technique is related to the use of natural stock pond water in that it uses soil to derive natural organic and inorganic nutrient sources. The process may comprise (1) adding soil to distilled water, then steaming the water to release nutrients (Andersen, 2005); (2) adding soil, soil extracts, or nutrient chemicals to distilled or natural water (lake water or seawater) (Cevera, 1996), though it is uncommon for freshwater as noted above, and it may or may not be sterilized, and (3) adding garden soil to tap water, then autoclaving for 45 min. (Atlas, 2010). These techniques are artificial means of enhancing the water and do not provide the natural microbial flora that is found in established stock ponds where algae grows naturally.

The process disclosed herein uses natural water and processes it in a manner that sterilizes and releases a nutrient-rich aseptic medium. When this medium is coupled to a culturing process, it provides a mechanism for recycling the medium and commercializing the algae product.

Seawater has also been used to prepare algal culture media (WO2013058432, incorporated by reference herein). In some embodiments herein, seawater may be used as a source of the aqueous component to prepare a conditioned culture medium. Other researchers have preferred fresh water sources for preparation of an algal culture medium (WO2010089762, incorporated by reference herein). Any cultured, open or closed water source may be used, including brackish water and wastewater. Where a cultured stock pond is used as a water source, the “natural flora” of the cultured stock pond may comprise algal cultures and other flora that have been previously cultivated or added to the cultured stock pond by an operator. Wastewater includes municipal, animal and industrial runoff that is unsuitable for drinking or plant crop cultivation. It is envisioned that a waste effluent stream or other forms of wastewater containing endogenous active flora may be used as a starting water source.

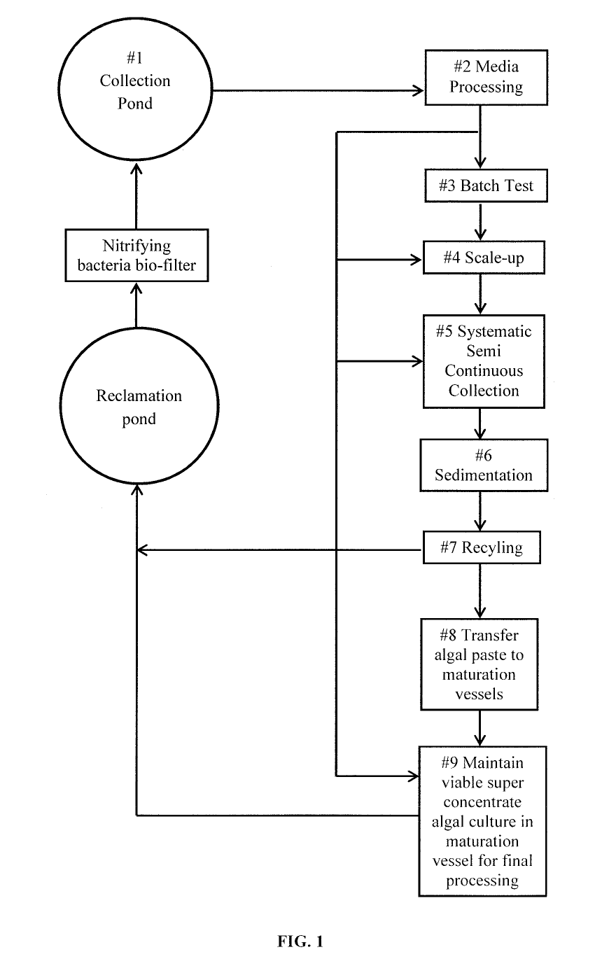

FIG. 1 is a process diagram showing one embodiment of a process for culturing algae. With reference to FIG. 1, the process may comprise: 1) obtaining water from a collection pond; 2) processing the pond water to make a conditioned culture medium; 3) conducting batch testing of algae grown in the conditioned medium; 4) progressive scale-up of the cultures to larger containers; 5) systematic semi-continuous collection of algal samples, with culture medium supplementation to maintain tank volume; 6) sedimentation and recovery of algal biomass; 7) recycling of algal biomass media; 8) transferring algal paste to maturation vessels; 9) maintaining viable super concentrate algal culture in maturation vessels for final processing; and return of reclaimed media to a reclamation pond.

It has been discovered that culture of algae using the methods and culture medium disclosed herein allowed the applicant to maintain a viable super concentrate culture for extended periods, without culture crash. In some applications, for example, an algae culture averaging 18 g/L (dry weight) was maintained for several months, with no measurable loss of viability. The typical algae culture density using standard media in commercial applications such as open ponds is about 1 g/L (dry weight) (Schenk, 2008, incorporated by reference herein). Batch testing may be eliminated in some embodiments, such as where such testing has previously been conducted on a similar processed pond water sample. In certain embodiments, both batch testing and scale-up may be bypassed. As will be appreciated by the person of skill in the art, other steps of the overall culturing process may be bypassed, depending on the conditions and needs of the operator. A nitrifying bacteria bio-filter may be used in some embodiments (see FIG. 1) to allow processed algae media to be returned to the environment. The use of a bio-filter reconditions the media and allows it to be returned to the collection pond so media processsing can begin again.

### EXAMPLES

The following examples are included to illustrate certain embodiments of the invention. These examples should not be construed as limitations to the claims. It should be appreciated by those of skill in the art that the techniques disclosed in the following examples represent specific approaches used to illustrate certain modes of practice. However, those of skill in the art will appreciate that many changes can be made in these embodiments without departing from the spirit and scope of the invention.

### Example 1

### Preparation of PTS Microalgae Culture Medium

Multiple culture tests were conducted on Scenedesmus dimorphus to develop a conditioned culture medium (PTS) to optimize algae growth conditions and to increase the organism's growth kinetics, resulting in significantly increased culture density and product yield.

Natural pond water samples obtained from regions near Houston, Tex. were autoclaved for 55 minutes at 121° C. and 13 psi (670 mm Hg) in Erlenmeyer flasks. Samples were prepared in 1 L volumes at a time to ensure adequate heating, then strained to remove debris. The heating process causes microorganisms in the water sample to be lysed, releasing their internal contents. These cellular factors, along with amino acids and other proteins, are absorbed by the algal cells in culture, minimizing the stress and energy needed for cellular metabolism.

Comparative testing of the conditioned medium against commercial culture media was performed. Two photobioreactors (PBRs) were inoculated with algae for observation over a period of time. Within 24 hours, visible growth was observed in the conditioned culture media but not in the control media. Within three days, the culture grown in the control media crashed, while the culture grown in the conditioned culture media continued to grow without further attention.

In small-scale samples, no additional additives or reagents were needed to maintain a viable culture using the conditioned media. In larger-scale 150 liter (40 gallon) tanks, light, nitrate, and phosphate were determined to be limiting factors in continued growth. Potassium nitrate and potassium phosphate monobasic were thus added and maintained above 40 ppm (nitrate) and 10 ppm (phosphate). Once these were added, cellular density increased dramatically and remained substantially at the same high density for several months. The conditioned culture medium successfully eliminated the problem of culture crash and significantly reduced the amount of labor-intensive maintenance required to keep an algae culture viable.

The algae culture medium was then evaluated to determine if it would support the growth of a variety of algae. The medium allowed approximately 40 different species and strains of algae to be successfully cultured, including both fresh and saltwater species.

### Example 2

### Algae Culture Process

An algae culturing process was designed to optimize growth of algae, allow long-term maintenance of an algae culture under controlled conditions, and allow for robust recovery of algal biomass samples for use in various industrial applications. The general process diagram for the algae culture process is shown in FIG. 1.

Collection and Media processing. Water was collected from a natural stock pond containing algae and other living organisms. The water was processed in three steps: 1) autoclaving at 121° C. at 13 psi (670 mm Hg) for a sufficient amount of time to eliminate bacteria, viruses, and spores); 2) straining the resulting solution using three coffee filters to form one multi-layer filter to remove particulates; and 3) addition of phosphate and nitrate to maintain culture chamber concentration of nitrate greater than 40 ppm and phosphate greater than 10 ppm, or at other levels determined by specific testing, depending on the algal species. The addition of phosphate and nitrates is timed specifically to the algal species and is based on batch testing on the initial growth. Depending on this testing, phosphate and nitrate were added either prior to inoculation or a specific period of time after inoculation for the scale up procedure.

Batch testing. Batch testing was conducted to determine growth kinetics and the optimal parameters for culture scale up under controlled lighting. These tests were conducted because of prior observed morphological and phenotypic changes of algae in the conditioned culture medium that allowed uncharacteristic growing conditions (e.g., growth in very high pH, unusual lighting conditions, or, as in the case of Nannochloropsis, transition from a salt water to fresh water condition).

Scale-up. Once growth parameters were determined along with algae growth response to phosphate and nitrate, cultures were scaled-up to chemostat-like conditions under controlled lighting. The scale up process began using small tanks and moved to successively larger tanks to allow a semi-continuous harvest, ultimately using standard round 150 liter (40 gallon) tanks. Scale-up was conducted in transparent containers in indoor, controlled conditions at room temperature (approximately 25° C.). There was no bubbling in these tanks, but a stirring/agitation apparatus (e.g., two bladed propeller) was used to promote aeration.

Systematic semi-continuous collection process. The appropriate volume and rate of algal solution removal was determined for the harvest process. As algal samples were removed from the tank, culture media was replaced to maintain a consistent solution volume and the concentration of phosphate and nitrates were monitored under controlled lighting. The collected algal samples were transferred into sedimentation apparatuses (environ shakers) to allow algal cells to settle out of solution, under controlled lighting. Culture media was drained and/or siphoned from the settled alga paste. The recovered culture media was recycled through a reclamation pond and nitrifying bacteria biofilter for later reuse.

Maturation vessels. The alga paste was transferred to maturation vessels to super-concentrate the algae and maintain the viability of existing cultures by addition of fresh media, with agitation under controlled lighting.

Maintaining super-concentrated alga cultures. Algae were maintained in maturation vessels until ready for final processing, with systematic medium exchange under controlled lighting. During this stage, processing can occur through media manipulation and/or other stressing techniques, because of the relative ease of separating algae from the aqueous solution.

### Example 3

### Comparative Yield of Algae Grown in Conditioned Culture Medium

The conditioned algae culture medium was tested against F2, an industry standard media. Careful monitoring of pH levels, cell morphology, and cell density under various conditions led to the development of a medium (PTS) that allowed algae culturing in chemostat-like conditions and resulted in a nearly eight-fold increase in cell density relative to cultures grown in F2. A semi-continuous, multi-step harvest process provided a means whereby the PTS medium could be separated from the algal mass and recycled.

The result of these modified culture techniques was a highly-concentrated algal culture that yielded a final dry weight average of 18 grams per liter of solution, whereas open ponds typically yield 1 gram dry weight per liter of solution, demonstrating that this process is much more efficient than traditional culture methods.

### Example 4

### Validation of PTS Conditioned Culture Media Against Standard Media

The conditioned culture media was tested against an industry standard media to evaluate growth kinetics and culture viability.

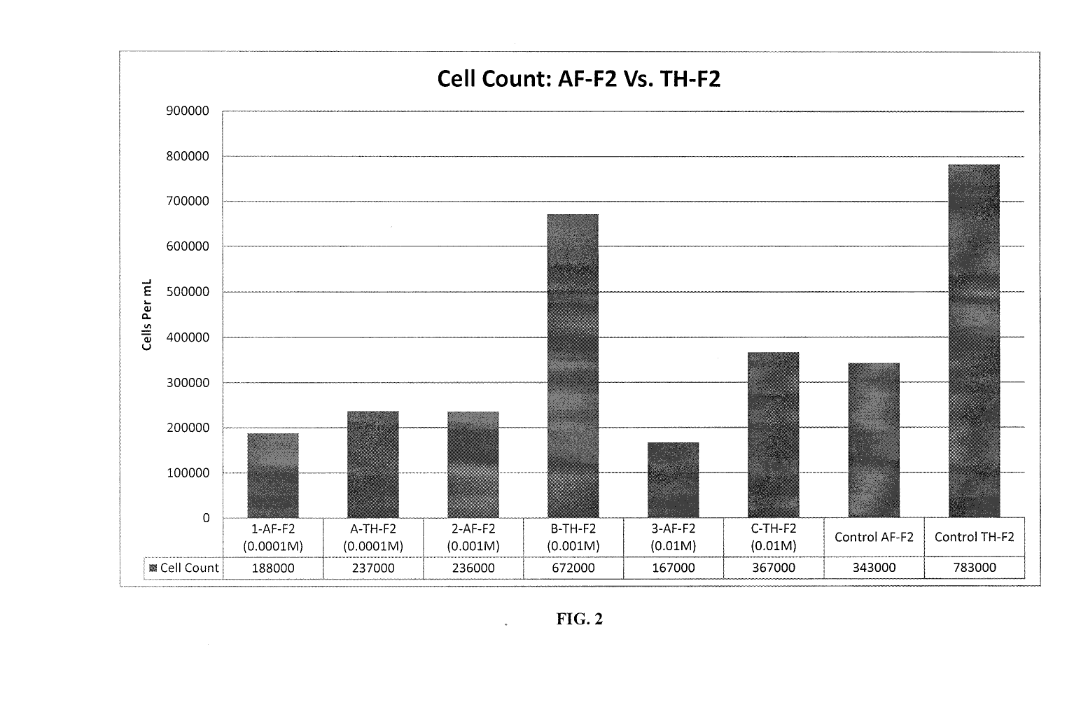

F2A6-F2A7 (F2) Microalgae Grow Mass Packs concentrate from Pentair Aquatic Eco-Systems (Sanford, N.C.) was mixed with either a standard culture media (designated AF) (n=3) or with the conditioned culture media described in Example 1 hereinabove (designated TH) (n=3). F2 concentrate was mixed at a ratio of 10 mls of F2 to 25 L distilled water and designated AF-F2. Conditioned culture media was autoclaved pond water in which 3.2 mL of F2 concentrate was added to 8 L of autoclaved pond water and designated TH-F2. See FIG. 2.

Starting media pH was adjusted using sodium phosphate (pH 8.00) at different molar concentrations (0.0001 M, 0.001 M, and 0.01 M), except for control samples which did not contain pH buffer. All tests were performed in batch (i.e., once parameters were set and medium inoculated, no further adjustments to reagents or conditions occurred). Control samples did not have buffer added.

FIG. 2 illustrates the growth reaction at the end of 19 days, in terms of cell density (cells/mL). Unexpected growth results were seen, in which B-TH-F2 had minimal pH buffering and Control TH-F2 had zero pH buffering control, but were the only two viable cultures left remaining when the test ended. The test was discontinued when algae cultures grown using only conventional media crashed.

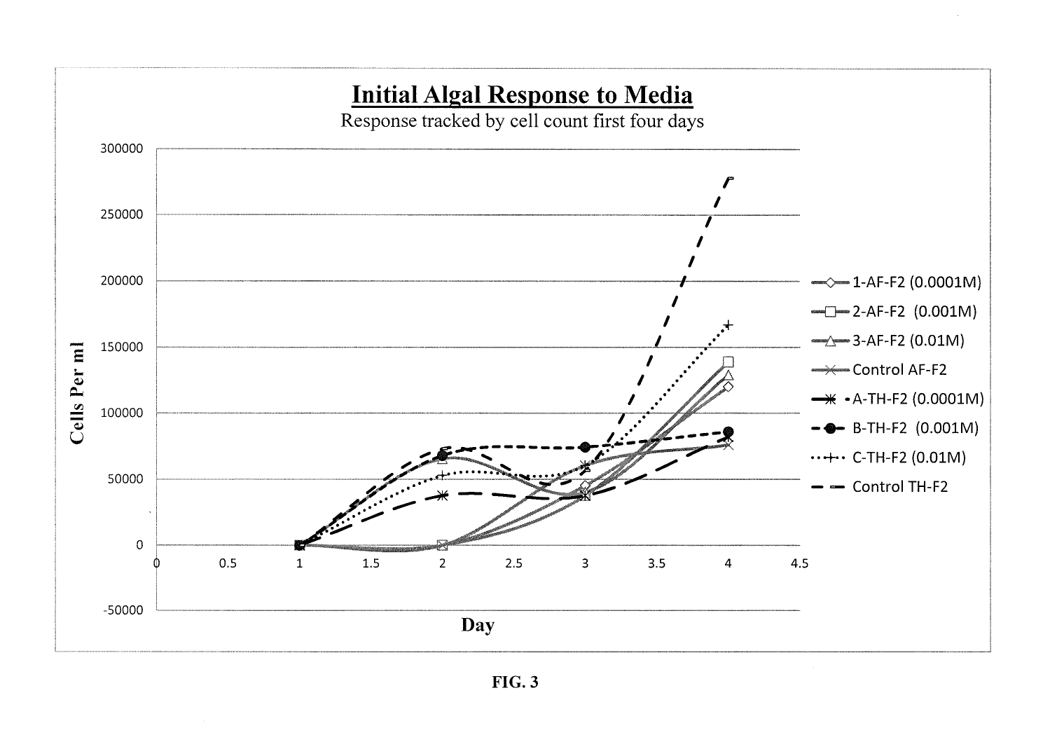

FIG. 3 illustrates the same experiment in which cell density was evaluated daily over four days. As is apparent, the reaction time for initial growth of algae grown in conditioned culture medium was substantially faster than conventional media.

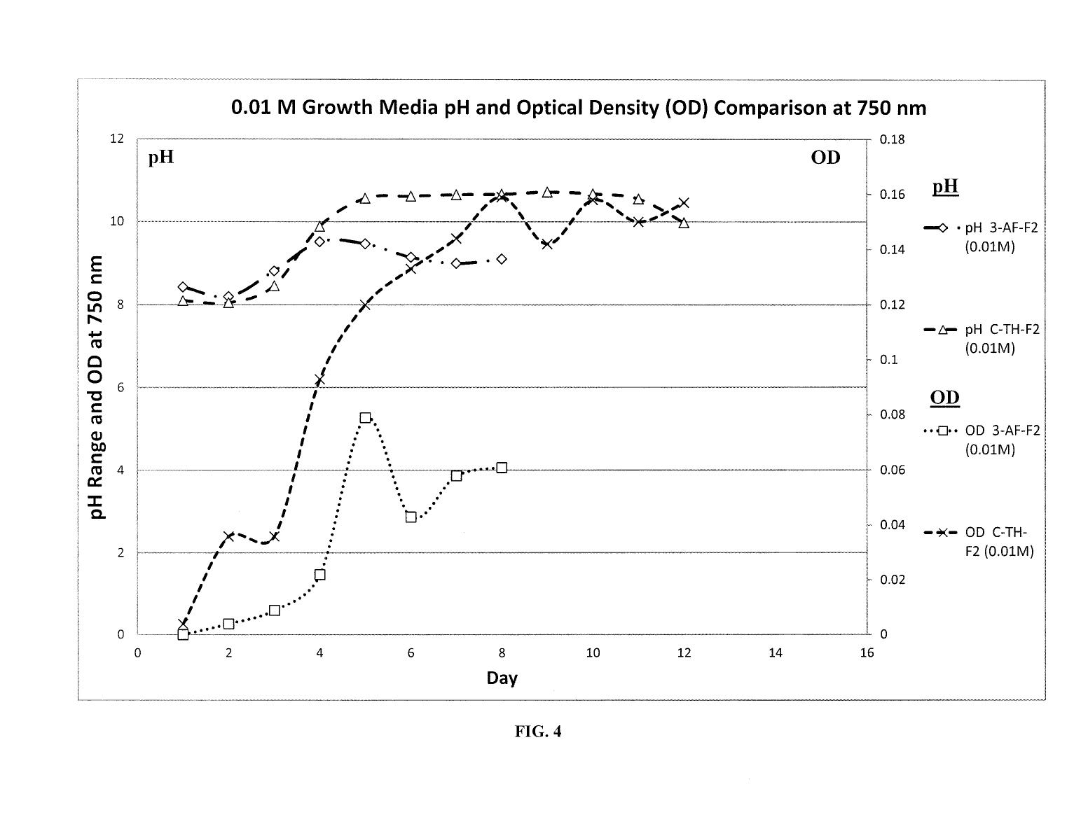

The growth of algae using conditioned culture media was evaluated as a function of pH. Algae was cultured substantially as described above, but pH was allowed to rise in all samples. Cell density was evaluated daily, and compared to the pH of the starting growth media. FIG. 4 shows algae cell density (OD₇₅₀) at varying growth media pH levels. The visible health and cellular density in the conditioned culture media (TH-F2) outpaced and continued viability far longer than industry standard F2 medium (AF-F2), as shown in FIG. 4. Algae grown using the conditioned culture media unexpectedly showed higher growth rates at a pH of approximately 10.0, in comparison to the normal optimal range of pH 8.2-8.7.

### Example 5

### Conditioned Culture Media Extends Viability of Algae Culture

The conditioned culture medium extended the viability of the algae culture significantly longer than the standard culture medium. At 14 days after inoculation, only one sample of the standard culture (2AF-F2), one sample of the conditioned control culture (BTH-F2), and one control sample (TH-F2) remained viable with algal growth, though one sample (2AF-F2) showed visible signs of failing (i.e., discoloration). At 17 days after inoculation, only BTH-F2 and control TH-F2 remained viable. Samples B-TH-F2, C-TH-F2, and control TH-F2 displayed significantly higher cells counts at day 5 than any algae grown in standard culture media.

### Example 6

### Scaled Algae Culture Using PTS Conditioned Culture Media

Algae was again cultured in PTS Conditioned Culture Media in a scaled-up process involving larger volumes and evaluating the effects of buffering agents on growth. Previous experiments suggested that non-buffered control media outperformed media buffered with sodium phosphate. Rather than sodium phosphate, sodium bicarbonate was used as the buffer. While non-buffered media successfully supported algal growth, two attempts to culture algae with sodium bicarbonate buffer failed.

Although pH is fairly easy to regulate in small batch samples, it can be difficult to regulate in larger samples. When pH was observed to be climbing in scale-up experiments, attempts to control the pH were increased. It was surprisingly discovered, however, that the algae grew best in pH ranges of pH 9.5-pH 10.2 using the conditioned culture medium disclosed herein. The algae survived in this conditioned medium without pH control assistance, but could not do so in the F2 medium without the conditioned media. Thus, the conditioned culture media displays an inherent buffering capacity that supports growth at much higher pH ranges than typically seen in microalgae culture.

It appears that the heating step used to prepare the conditioned culture medium releases cellular content rich in proteins, hormones, growth factors, and amino acids which have a buffering effect that prevents the pH from going significantly higher than pH 10.5. Further, despite the fairly large culture volumes, the microalgae cultured using the conditioned culture medium requires minimal maintenance, generally only requiring periodic removal and addition of 6 or 9 L of algae and new medium in 150 liter (40 gallon) culture tank.

### Example 7

### Multistage Harvesting Method

A chemostat-like approach to algae culturing was developed to allow for recovery of significant amounts of algal product while keeping the algal culture alive.

Rather than discarding high-density algae, algal samples (paste) were collected and just enough media was added to allow mixing. It was surprisingly discovered that the algal paste could be kept viable and growing for extended periods of time in this super concentrated form. A multistage harvesting technique thus entailed: (1) collecting a set volume of algae solution from the tank, (2) placing the collected volumes on a shaking apparatus overnight for sedimentation, (3) pouring off the separated medium into a collection vessel(s) for recycling, and (4) collecting algal paste in a secondary culturing vessel with only enough fresh medium to allow stirring (kept under a light), and (5) replacing the removed volume of solution with fresh medium.

Once scaled-up to 150 liter (40 gallon) tanks, the algal cultures are remarkably stable using this approach. Periodic removal of samples of algae solution allow recovery of biomass as needed. With the continued addition of media to bring the culture back to 150 liters (40 gallons, the pH shifted between the upper end of pH 8 to the upper end of pH 9. After stabilization of the culture, however, the pH hovered and maintained a range of the upper end of pH 9 to around pH 10-10.2 to this present time. The experimenters did not suffer a culture crash in over a year using this process, while the algal growth has remained dense and pH has remained consistently near 10.

This harvesting method allows continued growth and maturation of the cells (which may be later processed according to any desired product goal).

### Example 8

### Activating Sedimentation of Algae in Solution

Samples of algae solution were collected by flask and transferred to an environment shaker to encourage sedimentation. The discovery of a means to promote algae sedimentation from solution not only greatly simplified harvesting, it also opened up other areas, including recycling, medium swapping, and specific chemical manipulations. Recycling has two benefits: it greatly reduces water loss, and it encourages algal cell lysis in the recycling process, releasing specific hormones, growth factors, and proteins specific to that culture. Medium swapping allows a culture to be grown to a healthy state in a specific medium and then be switched into another medium at an appropriate time, causing specific reactions that will encourage more efficient production of specific byproducts. Peculiar chemical additives can be added and monitored in a more controlled manner, such as potassium phosphate or potassium nitrate. The sedimentation process leaves a concentrated algal paste that is easily handled and transported.

### Example 9

### Rescue of Algae

During the scale up process, it was discovered that the experimental F2 additive and buffering agents were detrimental to the algae culture. An attempt was made to rescue late-growth stage cultures from an imminent crash. Initially, potassium phosphate and potassium nitrate were added to the cultures without any noticeable improvement. Subsequently, seven remaining cultures totalling 15 L were deposited into a 150 liter (40 gallon) tank. These cultures were diluted by the addition of 30 L of fresh (PTS) conditioned culture media. Potassium nitrate and potassium phosphate monobasic were added and maintained as discussed previously herein.

The algal culture showed clear signs of recovery (i.e., visible growth) within a few days, and an active, thriving algal culture was apparent only two weeks later. These results showed that, in combination with potassium phosphate and potassium nitrate, culturing in the conditioned culture medium promotes a substantial increase in algal culture density.

1. A culture medium for growing algae, the culture medium comprising:
water obtained from a stock pond having natural flora, wherein the water has been treated to release biotic and abiotic factors from the natural flora of the stock pond; and
phosphates and nitrates.

2. The culture medium of claim 1, wherein the water has been treated by lysing the natural flora.

3. The culture medium of claim 2, wherein lysis comprises heating, sonication, chemical treatment, or light treatment.

4. A method of preparing the culture medium of claim 1, the method comprising:
obtaining water from a stock pond having natural flora;
treating the water to release biotic and abiotic factors from the natural flora of the stock pond;
and
adding phosphates and nitrates to form the culture medium.

5. The method of claim 4, wherein the stock pond is a cultured stock pond comprising algae.

6. The method of claim 4, wherein treating the water comprises lysing the natural flora of the stock pond.

7. The method of claim 5, wherein lysis comprises heating, sonication, chemical treatment, or light treatment.

8. The method of claim 4, further comprising dessicating the culture medium by removal of water to prepare a dry culture medium.

9. The method of claim 7, wherein the dry culture medium comprises a powder or paste.

10. A method of culturing algae, the method comprising:
introducing algae into the culture medium of claim 1; and
culturing the algae.

11. The method of claim 10, wherein treating the water comprises lysis of the natural flora.

12. The method of claim 10, wherein treating the water comprises heating, chemical treatment, light treatment, or sonication.

13. The method according to claim 10, further comprising harvesting the cultured algae.

14. The method of claim 13, wherein harvesting comprises sedimenting the algae.

15. The method of claim 10, wherein the algae are cultured in seawater, hypersaline water, desalination brine, brackish water, wastewater or freshwater.

16. The method of claim 10, wherein samples of algae are periodically removed from the algae culture, with addition of additional culture medium.

17. The method of claim 10, wherein one or more environmental parameters are altered to maintain optimal growth of the algae culture.

18. The method of claim 17, wherein the one or more environmental parameters are selected from the group consisting of pH; light intensity; light wavelength; nutrient concentration; nutrient balance; algal cell density; water temperature; concentration of dissolved gases; and ratio of dissolved gases.

19. The method of claim 10, wherein the algae culture is maintained at a pH between about 8.0 and about 10.0.

20. The method of claim 7, wherein:
lysis comprises heating;
the algae culture is maintained at a pH between 8.0 and 10.0; and
wherein the algae culture is maintained at a super-concentrated state.
